(19) United States
(12) Patent Application Publication
(10) Pub. No.: US 2009/0192326 A1
(43) Pub. Date: Jul. 30, 2009

(54) Preparation of sitagliptin intermediate

(76) Inventors: Nurit Perlman, Kfar Saba (IL); Marina Etinger, Nesher (IL); Valerie Niddam-Hildesheim, Kadima (IL); Mili Abramov, Givataim (IL)

Correspondence Address: KENYON & KENYON LLP, ONE BROADWAY, NEW YORK, NY, 10004, US

(21) Appl. No.: 12/291,925

(22) Filed: Nov. 13, 2008

Related U.S. Application Data
(60) Provisional application No. 61/003,033, filed on Nov. 13, 2007.
(60) Provisional application No. 61/072,854, filed on Apr. 2, 2008.
(60) Provisional application No. 61/130,843, filed on Jun. 3, 2008.

(51) Int. Cl. C07C 229/34

(52) U.S. Cl. 560/37

(57) ABSTRACT

Intermediate compounds in the synthesis of Sitagliptin, 3-amino-4-(2,4,5-trifluorophenyl)but-2-enoic acid alkyl ester, and amino protected-3-amino-4-(2,4,5-trifluorophenyl)but-2-enoic acid alkyl ester, and the stereoselective reduction of these compound to give Synthon I, or the amino-protected Synthon I, are provided.

### RELATED APPLICATIONS

This application claims the benefit of U.S. provisional application Nos. 61/003,033, filed Nov. 13, 2007, 61/003,553, filed Nov. 16, 2007, 61/068,653, filed Mar. 6, 2008, 61/072,854, filed Apr. 2, 2008, and 61/130,843, filed Jun. 3, 2008, hereby incorporated by reference in their entirety.

### FIELD OF THE INVENTION

The invention encompasses a process for the preparation of a Sitagliptin intermediate.

### BACKGROUND OF THE INVENTION

Sitagliptin, (3R)-3-amino-1-[9-(trifluoromethyl)-1,4,7,8-tetrazabicyclo[4.3.0]nona-6,8-dien-4-yl]-4-(2,4,5-trifluorophenyl)butan-1-one, has the following chemical structure:

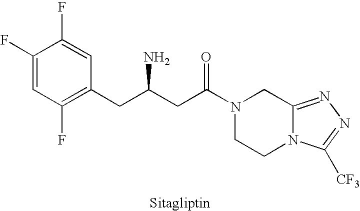

Sitagliptin is currently marketed in its phosphate salt in the United States under the tradename JANUVIA™ in its monohydrate form. JANUVIA™ is indicated to improve glycemic control in patients with type 2 diabetes mellitus. Sitagliptin phosphate is a glucagon-like peptide 1 metabolism modulator, hypoglycemic agent, and dipeptidyl peptidase IV inhibitor. Sitagliptin phosphate is described in PCT Publication No. WO 2005/003135.

Sitagliptin can be obtained by condensation of 2 key intermediates. The first intermediate is (3R)-amino-4-(2,4,5-trifluorophenyl)butanoic acid (“Synthon I”). Synthon I has the following formula:

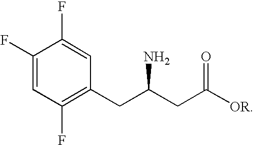

where R is H. The second intermediate is 3-(trifluoromethyl)-5,6,7,8-tetrahydro[1,2,4]triazolo[4,3-a]pyrazine (“Synthon II”), having the following formula:

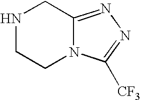

The following PCT Publications describe the synthesis of Sitagliptin using stereoselective reduction: WO 2004/087650, WO 2004/085661, and WO 2004/085378.

PCT Publication No. WO 2004/085378 refers to the synthesis of Sitagliptin intermediate (3R)-[protected-amino]-4-(2,4,5-trifluorophenyl)butanoic acid via stereoselective hydrogenation of a prochiral enamine, 3-Amino-1-(3-trifluoromethyl-5,6-dihydro-8H-[1,2,4]triazolo[4,3-a]purazin-7-yl)-4-(2,4,5-trifluorophenyl)but-2-en-1-one, using Rhodium complex with (R,S)-tert-butyl-Josipos ligand. PCT Publication No. WO 2004/087650 refers to the synthesis of Sitagliptin intermediate (3R)-[protected-amino]-4-(2,4,5-trifluorophenyl)butanoic acid via chiral reduction of 3-Oxo-4-(2,4,5-trifluorophenyl)-butyric acid with Ru—(S)-BINAP complex, followed by inversion of stereochemical center, achieved by Mitsunobu cyclization of (3S)-N-Benzoyloxy-3-hydroxy-4-(2,4,5-trifluorophenyl)butyramide. In PCT Publication No. WO 2004/085661, the reduction is performed on a substituted enamine, (S)-2-((Z)-4-(3-(trifluoromethyl)-5,6-dihydro-[1,2,4]triazolo[4,3-a]pyrazin-7(8H)-yl)-1-(2,4,5-trifluorophenyl)-4-oxobut-2-en-2-ylamino)-2-phenylacetamide with PtO₂.

U.S. Pat. No. 6,699,871 refers to the synthesis of the Sitagliptin intermediate (3R)-[protected-amino]-4-(2,4,5-trifluorophenyl)butanoic acid by using diazomethane, which is a very dangerous and explosive reagent, and can not be used in industrial scale. Additionally, (S)-2,5-dihydro-2-isopropyl-3,6-dimethoxypyrazine is used as the starting material and leads to high costs.

Hsiao et al, HIGHLY EFFICIENT SYNTHESIS OF β-AMINO ACID DERIVATIVES VIA ASYMMETRIC HYDROGENATION OF UNPROTECTED ENAMINES, JACS, 2004, 126, 9918-19, disclose the asymmetric hydrogenation of unprotected enamines with metal-ligand complexes, including ((S)-BINAP)RuCl₂. A yield of only 0.9 percent was obtained with ((S)-BINAP)RuCl₂ and 90 psig hydrogen over a period of 18 hours at 50° C.

### SUMMARY OF THE INVENTION

The present invention provides intermediate compounds in the synthesis of Sitagliptin: 3-amino-4-(2,4,5-trifluorophenyl)but-2-enoic acid alkyl ester, and amino protected-3-amino-4-(2,4,5-trifluorophenyl)but-2-enoic acid alkyl ester, and the stereoselective reduction of these compound to give Synthon I, or the amino-protected Synthon I, which are key intermediates in the preparation of Sitagliptin.

### DETAILED DESCRIPTION OF THE INVENTION

As used herein, the term “alkyl” refers to C₁-C₆ hydrocarbons. Preferably, the C₁-C₆ hydrocarbon is methyl or ethyl.

As used herein, the term optically pure refers to a sample of an optically active compound, comprising at least 90% percent of the predominant enantiomer.

As used herein, the term “room temperature” refers to a temperature of about 20° C. to about 35° C., more preferably about 25° C. to about 35° C., more preferably about 25° C. to about 30° C., and most preferably about 25° C.

The present invention encompasses a process for the preparation of a Sitagliptin key intermediate, 3-amino-4-(2,4,5-trifluorophenyl)butanoic acid, alkyl ester via enamine reduction. This synthesis gives high stereoselectivity.

In one embodiment, the present invention encompasses a process for preparing 3-amino-4-(2,4,5-trifluorophenyl)butanoic acid, alkyl ester (“Synthon I”-alkyl ester), comprising reducing 3-amino-4-(2,4,5-trifluorophenyl)but-2-enoic acid alkyl ester of the following formula:

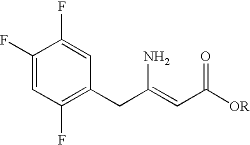

wherein R is C₁-C₆ alkyl (such as methyl, ethyl, iso-propyl and tert-butyl), C₆-C₁₂ aryl, C₇-C₁₂ arylalkyl, or C₇-C₁₂ alkylaryl, in the presence of hydrogen source and a chiral catalyst to obtain Synthon I-alkyl ester. Preferably, the reduction is stereoselective.

Preferably, the reduction reaction is carried out in the presence of an organic solvent. An acid may also be added to the reaction mixture. In a specific example, the process for preparing 3-amino-4-(2,4,5-trifluorophenyl)butanoic acid, alkyl ester (“Synthon I”-alkyl ester), comprises combining 3-amino-4-(2,4,5-trifluorophenyl) but-2-enoic acid alkyl ester with a chiral catalyst, and a hydrogen source, and optionally an acid, and in the presence of a solvent such as C₁-C₆ alcohol, or a C₁-C₆ fluorinated alkylalcohol. Preferably the molar ratio of the 3-amino-4-(2,4,5-trifluorophenyl) but-2-enoic acid alkyl ester and the chiral catalyst is from about 0.001% to about 5%. Preferably from about 3 ml to about 30 ml of alcohol are used per gram of the 3-amino-4-(2,4,5-trifluorophenyl) but-2-enoic acid alkyl ester. Typically, 3-amino-4-(2,4,5-trifluorophenyl)but-2-enoic acid alkyl ester used in the above process can be prepared using any method known in the art, for example, according to the reaction disclosed in Tetrahedron: Asymmetry 17 (2006), 205-209, and depicted in the following scheme:

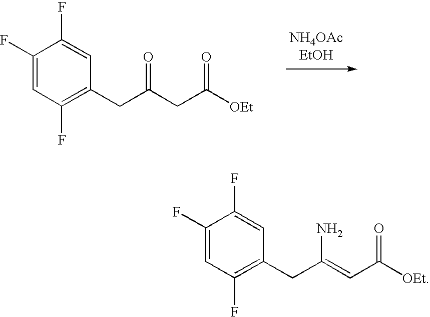

Preferably, the chiral catalyst is a complex Ru-BINAP. Preferably, the complex is formed from a mixture of a first metal complex and a chiral ligand. Example for the first metal complexes are [Ru(COD)X₂]ₙ (COD=1,5-cyclooctadiene, X=halogen, n=natural number. More preferably, the complexes are [Ru(COD)Cl₂]ₙ. Preferably X is F, Cl, or Br, more preferably X is Cl or Br, and most preferably, X is Cl.

Preferably The chiral ligand is (R or S)-2,2′-bis(diphenylphosphino)-1,1′-binaphthyl (BINAP), or derivatives thereof. More preferably, the ligand is (R)-2,2′-bis(diphenylphosphino)-1,1′-binaphthyl or (S)-2,2′-bis(diphenylphosphino)-1,1′-binaphthyl, and most preferably. Most preferably, the ligand is (R)-2,2′-bis(diphenylphosphino)-1,1′-binaphthyl.

Preferably, the solvent for the reaction is selected from the group consisting of C₁-C₆ alcohols, and C₁-C₆ fluorinated alkyl alcohols; more preferably, the solvent is a C₁-C₆ alcohol, or a C₁-C₆ fluorinated alkyl alcohol selected from the group consisting of: methanol, ethanol, isopropyl alcohol, and trifluoroethanol; preferably, ethanol or trifluoroethanol.

Preferably, the acid is an organic acid. More preferably, the organic acid is selected from the group consisting of: acetic acid, chloroacetic acid, propionic acid, and methanesulfonic acid. Most preferably, the organic acid is acetic acid. However, where the alcohol is a fluorinated C₁-C₆ alkyl alcohol, such as trifluoroethanol, no acid is needed.

Preferably, the reaction is carried out at about 5 to 7 bar of hydrogen pressure. More preferably, the pressure is about 5.5 to 6.5 bar. Preferably, the reaction mixture is maintained at a temperature of greater than 50° C. to about 140° C., preferably about 60° C. to about 100° C. (e.g. about 60° C. to 80° C.), and most preferably about 70° to about 90° C. Particularly, the reaction mixture may be maintained at a temperature of about 80° C. The reaction mixture is preferably maintained at this temperature for about 10 to 80 hours, preferably about 15 hours to about 60 hours, and more preferably about 15 hours to about 45 hours. Good results and high yields have been obtained maintaining the reaction fixture for a period of about 24 to about 45 hours.

If a fluorinated C₁₋₆ alcohol, such as trifluoroethanol is used, the reaction mixture is preferably maintained at a temperature of about 60° C. to about 100° C. (e.g. about 60° C. to 80° C.), and most preferably about 80° C. Preferably the mixture is maintained at this temperature for a period of about 10 to about 25 hours (e.g., about 10 to about 15 hours), more preferably. 12 to about 24 hours, and most preferably about 15-18 hours.

The 3-amino-4-(2,4,5-trifluorophenyl)butanoic acid, alkyl ester (“Synthon I”-alkyl ester) can be purified and recovered using any method known to those skilled in the art, for example, by extracting, washing, drying and evaporating.

Most preferably, the obtained 3-amino-4-(2,4,5-trifluorophenyl)butanoic acid, alkyl ester (“Synthon I”-alkyl ester) is optically pure. The ratio between the two enantiomers is preferably about 60% to about 100%, more preferably about 80% to about 100%, more preferably about 90% to about 100%, most preferably about 95% to about 99.8% (for example, the ration is 95.4:4.6 to about 99.5:0.5). Most preferably, the predominant enantiomer is the (3R) enantiomer of Synthon I-alkyl ester.

Preferably, the chemical purity of 3-amino-4-(2,4,5-trifluorophenyl)butanoic acid, alkyl ester according to HPLC is more that about 90%, preferably about 90% to about 100%, and most preferably about 90% to about 94% (preferably about 91.1% to 93.14%).

In a further embodiment of the present invention, there is provided a process for preparing 3-amino-4-(2,4,5-trifluorophenyl)butanoic acid, alkyl ester (“Synthon I”-alkyl ester comprising reducing 3-amino-4-(2,4,5-trifluorophenyl)but-2-enoic acid alkyl ester with a reducing agent (preferably a borohydride or a hydride reducing agent), in the presence of a chiral organic acid. The reaction is preferably carried out in the presence of an organic solvent. Preferably, this process for preparing 3-amino-4-(2,4,5-trifluorophenyl)butanoic acid, alkyl ester (“Synthon I”-alkyl ester) comprises combining 3-amino-4-(2,4,5-trifluorophenyl)but-2-enoic acid alkyl ester, a chiral organic acid, a reducing reagent and an organic solvent, and maintaining the reaction mixture for a sufficient period of time to obtain 3-amino-4-(2,4,5-trifluorophenyl)butanoic acid, alkyl ester. Preferably, the mixture is maintained for about 4 to about 24 hours. Preferably, the molar ratio of 3-amino-4-(2,4,5-trifluorophenyl)but-2-enoic acid alkyl ester, organic acid, and reducing reagent is from about 0.25 to about 0.4. Preferably, about 5 to about 40 ml of organic solvent is used per gram of 3-amino-4-(2,4,5-trifluorophenyl)but-2-enoic acid alkyl ester.

Optionally, the organic acid is optically pure. Most preferably, the organic acid is (R or S)-mandelic acid.

Preferably, the reducing reagent is a borohydride or hydride reducing reagent selected from the group consisting of sodium borohydride, sodium cyanoborohydride, lithium borohydride, and lithium aluminum hydride. More preferably, the reducing reagent is sodium borohydride. Preferably, the organic solvent is an ether, such as a C₄ to C₈ alkyl ether or a C₄ to C₈ cyclic ether. Most preferably the organic solvent is tetrahydrofuran.

Preferably, the mixture is stirred at a temperature of about −5° C. to about 30° C., more preferably at about room temperature, i.e., about 25° C. for about 8 to about 24 hours. The mixture is The mixture is stirred at this temperature range preferably for a period of about 30 minutes to about 20 hours, more preferably about 30 minutes to about 12 hours. More preferably the mixture is stirred at this temperature range for a period of about 1 to about 12 hours, and most preferably for about 12 hours.

Typically, the obtained 3-amino-4-(2,4,5-trifluorophenyl)butanoic acid, alkyl ester may be recovered and purified using any method known in the art, for example, by filtration.

Preferably, the obtained 3-amino-4-(2,4,5-trifluorophenyl)butanoic acid, alkyl ester is optically pure. Most preferably, the predominant enantiomer is the (3R) enantiomer of Synthon I-alkyl ester.

Typically, the enantiomers ratio of the obtained 3-amino-4-(2,4,5-trifluorophenyl)butanoic acid, alkyl ester is 92.2:7.8.

In another embodiment, the present invention encompasses a process for preparing Sitagliptin or salts thereof, comprising obtaining 3-amino-4-(2,4,5-trifluorophenyl)butanoic acid, alkyl ester by any of the methods described above, and further converting it to Sitagliptin or salts thereof.

In another embodiment the present invention encompasses 3-amino-protected-4-(2,4,5-trifluorophenyl)but-2-enoic acid alkyl ester of the following formula:

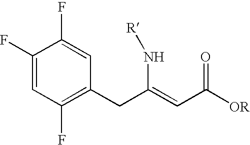

wherein R is alkyl, preferably a C₁-C₆ alkyl, more preferably C₁-C₄ alkyl, and most preferably methyl, ethyl, isopropyl and tert-butyl or C₆-C₁₂ aryl; and R′ is a C₁-C₄ alkoxycarbonyl, a C₁-C₄ haloalkoxycarbonyl, a C₆-C₁₂ benzyloxycarbonyl, tert-butoxycarbonyl (BOC), trityl, 9-fluorenylmethyl chloroformate (F-MOC), or a carbamate having the formula of —CO₂R² (CBZ, R²=Bn), —SO₂R³, or —PO(R³)₂, wherein R³ is an alkyl, an aryl, or an alkylaryl. Preferably R′ is BOC. Preferably, the 3-amino-protected-4-(2,4,5-trifluorophenyl)but-2-enoic acid alkyl ester is isolated.

In another embodiment, the present invention encompasses a process for preparing 3-tert-butoxycarbonylamino-4-(2,4,5-trifluorophenyl)but-2-enoic acid alkyl ester comprising converting the carbonyl group of 3-oxo-4-(2,4,5-trifluorophenyl)butanoic acid alkyl ester into a protected enamine functional group, according to the reaction.

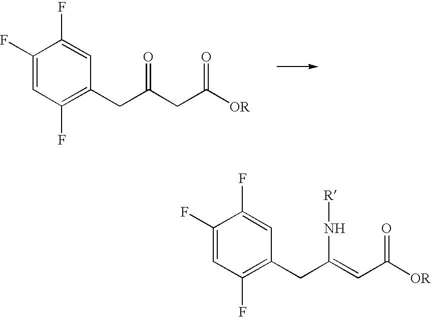

wherein R is alkyl preferably a C₁-C₆ alkyl, more preferably C₁-C₄ alkyl, and most preferably methyl, ethyl, isopropyl and tert-butyl, or C₆-C₁₂ aryls; and R′ is a C₁-C₄ alkoxycarbonyl, a C₁-C₄ haloalkoxycarbonyl, a C₆-C₁₂ benzyloxycarbonyl, tert-butoxycarbonyl (BOC), trityl, F-MOC, or a carbamate having the formula of —CO₂R² (CBZ, R²=Bn), —SO₂R³, or —PO(R³)₂, wherein R³ is an alkyl, an aryl, or an alkylaryl. Preferably R′ is BOC.

In another embodiment, the invention encompasses a process for preparing 3-tert-butoxycarbonylamino-4-(2,4,5-trifluorophenyl)but-2-enoic acid alkyl ester comprising reacting 3-oxo-4-(2,4,5-trifluorophenyl)butanoic acid alkyl ester with tert butyl carbamate. Preferably the reaction is carried out in the presence of a catalytic amount (0.01-0.1 equivalents) of organic acid. Preferably, the reaction is carried out in the presence of an organic solvent. Preferably, the process for preparing 3-tert-butoxycarbonylamino-4-(2,4,5-trifluorophenyl)but-2-enoic acid alkyl ester comprises combining tert-butyl carbamate with 3-oxo-4-(2,4,5-trifluorophenyl)butanoic acid alkyl ester, a catalytic amount (0.06 equivalents) of organic acid and an organic solvent (8 ml/gr). Preferably, the molar ratio of tert-butyl carbamate, 3-oxo-4-(2,4,5-trifluorophenyl)butanoic acid alkyl ester, and organic acid is from about 10 to about 100. Preferably, the organic solvent is used in an amount of from about 5 to about 20 ml per gram of 3-oxo-4-(2,4,5-trifluorophenyl)butanoic acid alkyl ester.

Tert-butyl carbamate used in the above process can be prepared using any method known in the art, for examples according to the procedure disclosed in Tetrahedron: Asymmetry, 12 (2001), 2989 and in Organic Synthesis, 48 (1968), 32.

Preferably, the organic acid is selected from the group consisting of p-toluenesulfonic acid, methansulfonic acid, and trifluoroacetic acid. More preferably, the organic acid is p-toluenesulfonic acid. Preferably, the organic solvent is a C₆-C₁₂ aromatic solvent, such as benzene, toluene, or chlorobenzene, or a halogenated C₁-C₆ alkane, such as methylene chloride; preferably the solvent is methylene chloride. Preferably, water removal is carried out during the reaction. Form example, water removal may be carried out by the addition of a drying agent, or by azeotropic distillation. Preferably, a drying agent is introduced to the reaction mixture. The drying agent may be selected from any drying agent known to the skilled in the art. Most preferably, the drying agent is a molecular sieve, more preferably, MS-4 Å (Molecular Sieves-4 Å). Optionally, the water may be removed from the reaction mixture by azeotropic distillation.

Preferably, 3-tert-butoxycarbonylamino-4-(2,4,5-trifluorophenyl)but-2-enoic acid alkyl ester is further recovered and purified by any method known in the art, for example, by evaporation and purification using HPLC techniques and/or crystallization.

In another embodiment, the present invention encompasses a process for preparing Sitagliptin or salts thereof, comprising obtaining 3-tert-butoxycarbonylamino-4-(2,4,5-trifluorophenyl) but-2-enoic acid alkyl ester as described above, and further converting it to Sitagliptin or salts thereof.

In another embodiment, the present invention encompasses a process for preparing the amino-protected group, 3-amino-protected-4-(2,4,5-trifluorophenyl) butanoic acid alkyl ester comprising reducing 3-amino-protected-4-(2,4,5-trifluorophenyl) but-2-enoic acid alkyl ester, in the presence of hydrogen and a chiral catalyst. Preferably, the reduction is stereoselective. Preferably, the reduction is carried out by a process as defined in any embodiment of the present invention. Preferred reagents, solvents, catalysts and conditions for this reduction are described above, and are also applicable to the reduction of 3-amino-protected-4-(2,4,5-trifluorophenyl)but-2-enoic acid alkyl ester. Preferably, the 3-amino-protected-4-(2,4,5-trifluorophenyl)butanoic acid alkyl ester is 3-tert-butoxycarobylamino-4-(2,4,5-trifluorophenyl)butanoic acid alkyl ester.

Preferably the reaction is conducted in the presence of a solvent such as C₁-C₆ alcohol or a fluorinated C₁₋₆ alkyl alcohol. Preferred metal complexes, chiral ligands, solvents and conditions are as described in any of the above embodiments for the reduction of 3-amino-4-(2,4,5-trifluorophenyl) but-2-enoic acid alkyl ester. In a specific example, the process for preparing the amino-protected group “Synthon I”, 3-tert-butoxycarbonylamino-4-(2,4,5-trifluorophenyl)butanoic acid alkyl ester comprises combining 3-tert-butoxycarbonylamino-4-(2,4,5-trifluorophenyl)but-2-enoic acid alkyl ester with a chiral catalyst, a hydrogen source and a C₁-C₆ alcohol. Preferably, the molar ratio of 3-tert-butoxycarbonylamino-4-(2,4,5-trifluorophenyl)but-2-enoic acid alkyl ester, a metal complex, and a chiral ligand is from about 5% to about 0.01%. Preferably about 3 ml to about 10 ml of the C₁-C₆ alcohol is used per gram of 3-tert-butoxycarbonylamino-4-(2,4,5-trifluorophenyl)but-2-enoic acid alkyl ester.

Preferably, the chiral catalyst is a complex Ru-BINAP. Preferably, the complex is formed from a mixture of a first metal complex and a chiral ligand. Example for the first metal complexes are [Ru(COD)X₂]ₙ (COD=1,5-cyclooctadiene, X=halogen, n=natural number. More preferably, the complexes are [Ru(COD)Cl₂]ₙ. preferably X is F, Cl, or Br, more preferably X is Cl or Br, and most preferably, X is Cl.

Preferably, the metal complex is composed of [Ru(COD)Cl₂]ₙ and BINAP.

The chiral ligand is (R or S)-2,2′-bis(diphenylphosphino)-1,1′-binaphthyl or (R or S)-2,2′-bis(diphenylphosphino)-1,1′-binaphthyl. Preferably, the ligand is (R)-2,2′-bis(diphenylphosphino)-1,1′-binaphthyl or (S)-2,2′-bis(diphenylphosphino)-1,1′-binaphthyl. Preferably, the ligand is (R)-2,2′-bis(diphenylphosphino)-1,1′-binaphthyl.

Preferably, the solvent is a C₁-C₆ alcohol or a fluorinated C₁-C₆ alcohol, and is more preferably a C₁-C₄ alcohol or a fluorinated C₁-C₄ alcohol, and is most preferably selected from the group consisting of: methanol, ethanol, isopropyl alcohol, and trifluoroethanol. More preferably, the alcohol is trifluoroethanol.

Preferably, the reaction is carried out in the presence of an organic acid. More preferably, the organic acid is selected from the group consisting of: acetic acid, chloroacetic acid, propionic acid, and methanesulfonic acid. Most preferably, the organic acid is acetic acid.

Preferably, the reaction is carried out at a hydrogen pressure of about 3 bar to about 8 bar, more preferably about 4 bar to about 7 bar, and most preferably about 5 to 7 bar, particularly at about 5 bar. Preferably, the reaction mixture is maintained at about 40° C. to about 100° C., more preferably about 60° C. to about 100° C., and most preferably about 60° C. to about 80° C. for about 10 to 80 hours, preferably about 20 hours to about 60 hours, and more preferably about 30 hours, to about 50 hours. Most preferably, the reaction is carried out at about 5 bar at 80° C. for about 40 hours.

The protected 3-amino-4-(2,4,5-trifluorophenyl)butanoic acid, alkyl ester (protected-“Synthon I”-alkyl ester) can be purified and recovered using any method known to the skilled in the art, for example, by extracting, washing, drying and evaporating.

Preferably, the obtained protected-3-amino-4-(2,4,5-trifluorophenyl)butanoic acid, alkyl ester is optically pure. Most preferably, the predominant enantiomer is the (3R) enantiomer of protected-Synthon I-alkyl ester.

In another embodiment, the present invention encompasses a process for preparing Sitagliptin or salts thereof, comprising obtaining 3-tert-butoxycarbonylamino-4-(2,4,5-trifluorophenyl)butanoic acid alkyl ester by any of the methods described above, and further converting it to Sitagliptin or salts thereof.

Sitagliptin can be prepared by other processes, such as coupling 3-protected-amino-4-(2,4,5-trifluorophenyl)butanoic acid with 3-(trifluoromethyl)-5,6,7,8-tetrahydro[1,2,4]triazolo[4,3-a]pyrazine hydrochloride to obtain (R)-3-protected-amino-1-(3-(trifluoromethyl)-5,6-dihydro-[1,2,4]triazolo[4,3-a]pyrazin-7(8H)-yl)-4-(2,4,5-trifluorophenyl)butan-1-one; and then removing the amino protected group to obtain Sitagliptin.

Preferably; 4-(3-(trifluoromethyl)-5,6-dihydro-[1,2,4]triazolo[4,3-a]pyrazin-7(8H)-yl)-1-(2,4,5-trifluorophenyl)-4-oxobutan-2-yl-carbamate is optically pure. Most preferably, the obtained coupling product is (R)-4-(3-(trifluoromethyl)-5,6-dihydro-[1,2,4]triazolo[4,3-a]pyrazin-7(8H)-yl)-1-(2,4,5-trifluorophenyl)-4-oxobutan-2-yl-carbamate.

Optionally, the 3-tert-butoxycarbonylamino-4-(2,4,5-trifluorophenyl)butanoic acid is cooled to a temperature of about −10° C. to about 25° C., more preferably about 0° C. in the presence of a first organic solvent; followed by the addition of Dicyclohexylcarbodiimide in a second organic solvent; introducing 3-(trifluoromethyl)-5,6,7,8-tetrahydro[1,2,4]triazolo[4,3-a]pyrazine hydrochloride, an organic base, and a catalyst to the reaction mixture; and recovering 4-(3-(trifluoromethyl)-5,6-dihydro-[1,2,4]triazolo[4,3-a]pyrazin-7(8H)-yl)-1-(2,4,5-trifluorophenyl)-4-oxobutan-2-yl-carbamate.

Preferably, the first and the second organic solvents are selected from the group consisting of aprotic solvent, such as dimethylformamide, tetrahydrofuran, and dichloromethane. Dimethylformamide is preferred. Preferably, a solution of Dicyclohexylcarbodiimide and Dimethylformamide is added drop-wise. Most preferably the catalyst is 4-Dimethylaminopyridine (“DMAP”). Suitable organic bases for this reaction are alkyl amines, preferably C₁-C₆ trialkyl amines, more preferably triethylamine, diisopropyl ethyl amine, and N methyl morpholine.

4-(3-(trifluoromethyl)-5,6-dihydro-[1,2,4]triazolo[4,3-a]pyrazin-7(8H)-yl)-1-(2,4,5-trifluorophenyl)-4-oxobutan-2-yl-carbamate can be recovered from the reaction mixture by any method known in the art, such as extraction, evaporation, filtration, and re-crystallization.

The amine protected group (such as BOC) can be removed by any method known in the art. For example, by reacting with an acid (such as a mineral acid). In a preferred embodiment the deprotection of the amine protecting group is carried out by introducing a solution of concentrated hydrochloric acid into a solution of tert-butyl-4-(3-(trifluoromethyl)-5,6-dihydro-[1,2,4]triazolo[4,3-a]pyrazin-7(8H)-yl)-1-(2,4,5-trifluorophenyl)-4-oxobutan-2-yl-carbamate and an organic solvent selected from a group consisting of C₁-C₆ alcohols, most preferably, the organic solvent is iso-propanol; heating the reaction mixture at about 40° C. for a sufficient period of time. Most preferably the reaction mixture is heated at about 25° C. to about reflux, preferably to about 40° C. for about 1 hour to about 24 hours, preferably an hour to about 5 hours, and more preferably about 2 hours; basifying the reaction mixture with an inorganic base, such as alkali bicarbonate, alkali carbonates, or alkali hydroxides, for example, sodium hydroxide; and recovering Sitagliptin.

Sitagliptin may be recovered from the reaction mixture using any known method, such as evaporation, extraction, and filtration.

Having described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. It will be apparent to those skilled in the art that many modifications, both to materials and methods, may be practiced without departing from the scope of the invention.

### EXAMPLES

### HPLC Method Conditions for Chromatographic Purity

Column: Luna C18 (2), 5 μm, 250 mm×4.6 mm (Phenomenex) or equivalent

### Solvent A: Acetonitril; Solvent B: 10 mM KH2PO4 (1.36 g) and 10 mM (0.4 g) NaOH in

Water (1 L) adjusted to pH 7.9 with 0.25 M H3PO4

Gradient: 0 min-45% A/55% B, 30 min-80% A/20% B, 35 min-80% A/20% B, 40 min 45% A/55% B, 45 min 45% A/55% B;
1.0 mL/min, PDA/UV at 210 nm, 30° C.

### Chiral HPLC Method Conditions:

Column: Chiralpak AD-H, 5 μm, 150 mm×4.6 mm (Daicel Chemical Ind., Cat. No. 19324) or equivalent;
5% EPA/97% Hexane (v/v);
1.0 mL/min, 35° C.; PDA/UV at 270 nm

### Example 1

### Preparation of 3-amino-4-(2,4,5-trifluorophenyl)but-2-enoic Acid Ethyl Ester

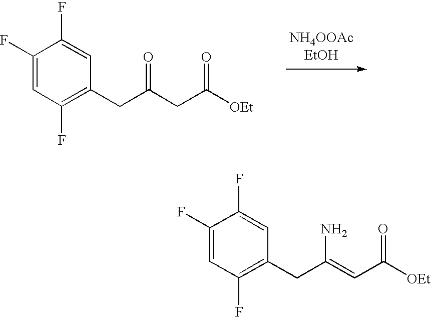

A mixture of 3-oxo-4-(2,4,5-trifluorophenyl)butanoic acid alkyl ester (7.0 g, 0.027 mol) and ammonium acetate (10.4 g, 0.135 mol) in absolute ethanol (80 mL) was refluxed for 2 hours, evaporated and diluted with ethyl acetate (100 ml). The precipitate was filtered off and the filtrate was evaporated to give white solid 3-amino-4-(2,4,5-trifluorophenyl) but-2-enoic acid alkyl ester which was used directly without further purification in the preparation of 3-amino-4-(2,4,5-trifluorophenyl)butanoic acid alkyl ester (shown in example 2).

¹H NMR (CDCl₃, δ): 1.25 (t, 3H), 3.39 (3, 2H), 4.08 (q., 2H), 4.55 (s, 1H), 6.85-7.15 (m, 2H).

### Example 2

### Preparation of 3-amino-4-(2,4,5-trifluorophenyl)butanoic Acid Ethyl Ester (“Synthon I”)

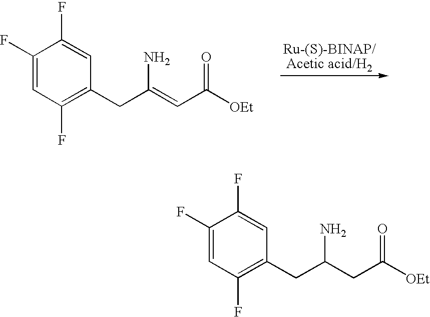

A mixture of 3-amino-4-(2,4,5-trifluorophenyl)but-2-enoic acid ethyl ester (1.25 g, 4.9 mmol), acetic acid (0.29 g, 4.9 mmol), [Ru(COD)Cl₂]ₙ (0.0138 g, 0.049 mmol) and (S)-BINAP (0.049 mmol, 1 mol %) in absolute ethanol (20 mL) was hydrogenated at 5.5 bar and 80° C. for 24 hours. The mixture was evaporated and the residue was treated with methyl tert butyl ether (MTBE)(10 mL) and 10% citric acid (10 mL). The MTBE layer was discarded, the aqueous. The layer was basified with NaHCO₃ and extracted with MTBE. Evaporation of the MTBE layer gave 3(S)-amino-4-(2,4,5-trifluorophenyl) butanoic acid ethyl ester (0.55 g, 43% yield), with 93.14% purity by HPLC, as a mixture of enantiomers in the ratio of about 95.4:4.6.

### Example 3

### Preparation of 3-amino-4-(2,4,5-trifluorophenyl)butanoic Acid Ethyl Ester (“Synthon I”)

250 ml stainless steel autoclave was charged with 33 g of 3-amino-4-(2,4,5-trifluorophenyl) but-2-enoic acid ethyl ester, 0.793 g of (R)-BINAP, 0.357 g of Ru(COD)Cl₂ and purged with N₂. Then, 165 ml of degassed CF₃CH₂OH was added. The mixture was stirred under N₂ atmosphere for 30 min at 25° C. and then hydrogenated at 80° C. and 5.5-6.5 bar for 17 hours.

The mixture was evaporated under reduced pressure. The obtained oily residue was dissolved in the mixture of 10% aq. Citric acid (450 ml) and MTBE (350 ml). The organic layer was separated. The aqueous layer was extracted with MTBE (100 ml×2); the pH was adjusted to 10 by addition of 10% aq. Na₂CO₃ (600 ml) and the solution was extracted with MTBE (100 ml×5). The combined extract was dried over Na₂SO₄, filtered through SiO₂ (15 g) and evaporated under reduced pressure to give 27.75 g of 3(R)-amino-4-(2,4,5-trifluorophenyl)butanoic acid ethyl ester as oil (purity 91.1%)

### Example 4

### Preparation of 3-amino-4-(2,4,5-trifluorophenyl) Butyric Acid Ethyl Ester (“Synthon I”)

2.43 gr sample of (S)-mandelic acid was dissolved in 10 ml tetrahydrofuran (THF) and cooled in ice bath. Then 0.43 gr NaBH₄ was added gradually, and the obtained mixture was stirred for 30 minutes. Then, 1 gr of 3-amino-4-(2,4,5-trifluorophenyl)but-2-enoic acid ethyl ester was dissolved in 2 ml of THF and added to the NaBH₄-mandelic acid mixture. The white mixture was stirred at room temperature over night. The mixture was analyzed by HPLC to give 3-amino-4-(2,4,5-trifluorophenyl)butanoic acid ethyl ester, (purity 17.9%) with a ratio of enantiomers of about 92.2 to 7.8.

### Example 5

### Preparation of 3-amino-4-(2,4,5-trifluorophenyl)butanoic Acid, Ethyl Ester (Racemic Mixture)

Sodium borohydride (1.12 g, 0.03 mol) was added carefully with portions to acetic acid at 15° C. to 20° C. (strong, exothermic reaction). 3-amino-4-(2,4,5-trifluorophenyl)but-2-enoic acid ethyl ester (2.6 g, 0.01 mol) was added to the prepared mixture at 20° C., and the resulting mixture was stirred for 1 hour at 20° C. to 25° C. Acetic acid was evaporated, the residue was dissolved in methylene chloride (100 mL), and washed with an aqueous saturated solution of NaHCO₃ to pH 10-11. The organic layer was dried over Na₂SO₄, filtered, and evaporated to give yellowish oil (1.45 g, 56% yield). The oil was treated with a solution of HCl/EtOH and evaporated to give yellowish oil (1.7 g), which solidified with time

### Example 6

### Preparation of 3-tert-butoxycarbonylamino-4-(2,4,5-trifluorophenyl)-but-2-enoic Acid Methyl Ester

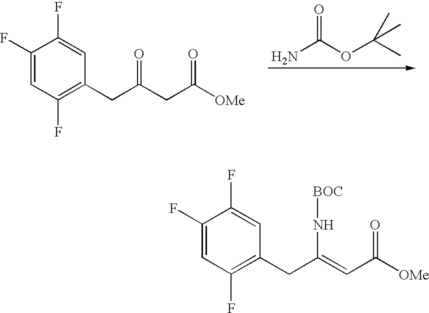

A solution of di-tert-butyl dicarbonate (21.82 g, 0.1 mol) in methanol (50 mL) was added to an 8 N solution of ammonia in methanol (50 mL) over a period of 1 hour at 0° C. The mixture was stirred at 25° C. for 15 hours and concentrated in vacuo. Hexane (100 mL) was added, the resulting mixture was stirred at 65° C. for 30 minutes and cooled to 0° C. The precipitate was collected by filtration to give tert-butyl carbamate as white crystals (10.4 g, 89%). NMR confirms the structure.

A mixture of 3-oxo-4-(2,4,5-trifluorophenyl)butanoic acid methyl ester (2.46 g, 0.01 mol), tert-butyl carbamate (1.79 g, 0.015 mol), p-toluenesulfonic acid (p-TSA) (0.1 g) and MS-4 Å (3.0 g) in methylene chloride (20 mL) was stirred overnight at 25° C. The mixture was filtered, evaporated, and purified on silica gel (10 g). The product was crystallized from hexane to give 3-tert-butoxycarbonylamino-4-(2,4,5-trifluorophenyl)but-2-enoic acid methyl ester as white solid (1.87 g, 54.1%).

¹H NMR (CDCl₃, δ): 1.43 (s, 9H), 3.66 (s, 3H), 4.06 (s, 2H), 4.68 (s, 1H), 6.80-7.20 (m, 2H), 10.40 (s, 1H).

### Example 7

### Preparation of 3-tert-butoxycarbonylamino-4-(2,4,5-trifluorophenyl)butanoic Acid Methyl Ester

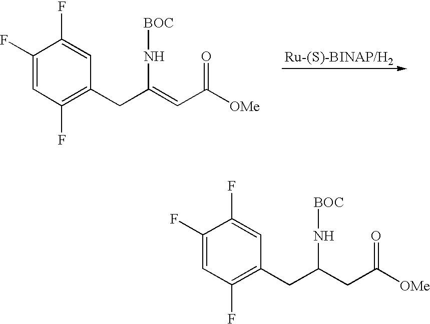

A mixture of 3-tert-butoxycarbonylamino-4-(2,4,5-trifluorophenyl)but-2-enoic acid methyl ester (1.5 g, 4.34 mmol), [Ru(COD)Cl₂]ₙ (0.0120 g, 0.043 mmol) and (S)-BINAP (0.0285 g, 0.043 mmol) in de-gassed methanol (22 mL) was hydrogenated for 40 hours at 80° C. and 5 bar. The mixture was evaporated, and the residue was purified on silica gel (30 g), hexane/ethyl acetate, to give 3-tert-butoxycarbonylamino-4-(2,4,5-trifluorophenyl)butanoic acid methyl ester (0.62 g, 41%) as white solid with 98.66% purity by HPLC and the ratio of enantiomers of about 66.2 to 33.8.

¹H NMR (CDCl₃, δ): 1.35 (s, 9H), 2.50 (d, 2H), 2.82 (d, 2H), 3.67 (s, 3H), 4.09 (m, 1H), 5.07 (d, 1H), 6.80-7.25 (m, 2H).

Un-reacted 3-tert-butoxycarbonylamino-4-(2,4,5-trifluorophenyl)but-2-enoic acid methyl ester was recovered from the reaction; as white solid in amount of 0.36 g (24.0%).

### Example 8

### Preparation of (R)-4-(3-(trifluoromethyl)-5,6-dihydro-[1,2,4]triazolo[4,3-a]pyrazin-7(8H)-yl)-1-(2,4,5-trifluorophenyl)-4-oxobutan-2-yl-carbamate

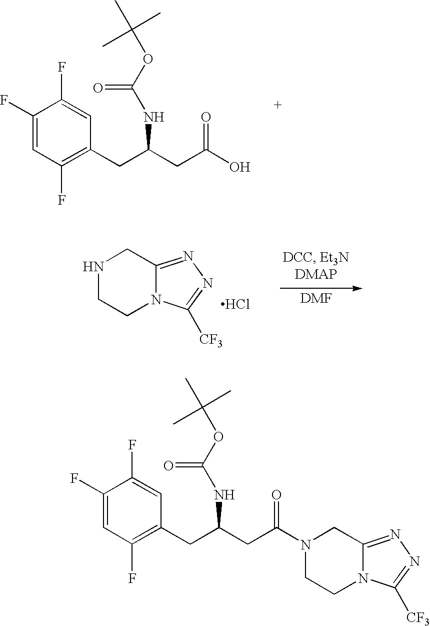

(3R)-3-tert-butoxycarbonylamino-4-(2,4,5-trifluoro-phenyl)-butyric acid (40 g, 0.12 mol) was dissolved in dimethylformamide (DMF)(240 mL) at room temperature while the reaction flask was under N₂, then, cooled with ice bath and stirred for 30 minutes. In a different flask, DCC (32.21 g, 0.16 mol) was dissolved in DMF (160 mL) to obtain a 200 mL solution. To the (3R)-3-tert-butoxycarbonylamino-4-(2,4,5-trifluoro-phenyl)-butanoic acid solution was added 70 mL from the DCC solution drop-wise, 3-(trifluoromethyl)-5,6,7,8-tetrahydro-[1,2,4]triazolo[4,3-a]pyrazine hydrochloride (32.94 g, 0.14 mol) and Et₃N (24.82 g, 0.24 mol). The reaction was stirred for 10 minutes, then, DMAP was added (8.8 g, 0.07 mol). The reaction was stirred for 2 hours, then, 65 mL of DCC solution was added drop-wise, and after another 1 hour of stirring in an ice bath, the last 65 mL of DCC solution was added drop-wise. The reaction was stirred at room temperature over night. The mixture was filtrated by vacuum filtration and washed with DMF 2×50 mL. The solvent was evaporated and EtOAc was added (1400 mL), the organic phase washed with 90 mL of 5% citric acid, 60 mL of 10% citric acid, and 100 mL of saturated NaHCO₃, dried over Na₂SO₄ and evaporated to yield a beige solid. The product was dissolved in IPA (300 mL) by heating to reflux. When the solution became clear-yellow the solution was cooled to room temperature and stirred over night. The product was isolated by vacuum filtration, washed isopropanol, and dried in a vacuum oven at 40° C. overnight to obtain tert-butyl (R)-4-(3-(trifluoromethyl)-5,6-dihydro-[1,2,4]triazolo[4,3-a]pyrazin-7(8H)-yl)-1-(2,4,5-trifluorophenyl)-4-oxobutan-2-yl-carbamate (52 g, 85% yield).

### Example 9

### Preparation of (3R)-3-amino-1-[9-(trifluoromethyl)-1,4,7,8-tetrazabicyclo[4.3.0]nona-6,8-dien-4-yl]-4-(2,4,5-trifluorophenyl)butan-1-one

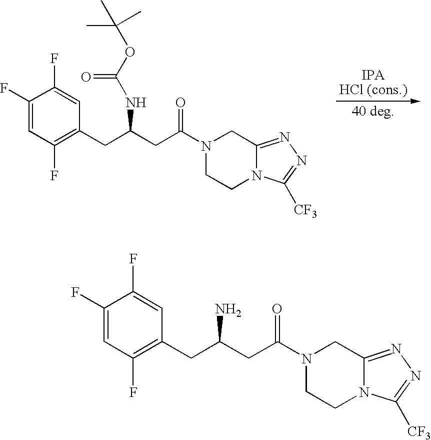

To a slurry of tert-butyl (R)-4-(3-(trifluoromethyl)-5,6-dihydro-[1,2,4]triazolo[4,3-a]pyrazin-7(8H)-yl)-1-(2,4,5-trifluorophenyl)-4-oxobutan-2-yl-carbamate (33.18 g, 0.065 mol) in IPA (3 vol., 100 mL) was added concentrated HCl (38 mL, 0.458 mol, 7 equiv.), and the reaction was heated at 40° C. for 2 hours. While heating, the solution became clear. The reaction cooled to room temperature, IPA was evaporated, MTBE (100 mL) was added, and then NaOH 16% was added drop-wise until PH˜12. The aqueous layer was extracted with MTBE (2×100 mL), and with a mixture of MTBE:isopropanol (10:1). The combined organic layer was washed with brine, dried over Na₂SO₄, filtered, and evaporated to yield colorless oil. Triturating with 50 mL MTBE at room temperature led to precipitation of white solid. The product was isolated by vacuum filtration, washed with methyl tert butyl ether, and dried in a vacuum oven at 40° C. overnight to obtain Sitagliptin (20.65 g, 77%).

What is claimed is:

1. A process for preparing 3-amino-4-(2,4,5-trifluorophenyl)butanoic acid alkyl ester (“Synthon I”-alkyl ester), comprising reacting 3-amino-4-(2,4,5-trifluorophenyl)but-2-enoic acid alkyl ester of the following formula: 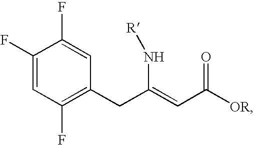
in the presence of hydrogen and a chiral catalyst to obtain 3-amino-4-(2,4,5-trifluorophenyl) butanoic acid alkyl ester;
wherein R is a C₁-C₆ alkyl, a C₆-C₁₂ aryl, a C₇-C₁₂ arylalkyl, or a C₇-C₁₂ alkylaryl; R′ is a hydrogen atom, a C₁-C₄ alkoxycarbonyl, a C₁-C₄ haloalkoxycarbonyl, a C₆-C₁₂ benzyloxycarbonyl, tert-butoxycarbonyl (BOC), trityl, F-MOC, or a carbamate having the formula of —CO₂R² (CBZ, R²═Bn), —SO₂R³, or —PO(R³)₂, wherein R³ is an alkyl, an aryl, or an alkylaryl; and
wherein the chiral catalyst is a complex of Ru-BINAP, wherein BINAP is (R)-2,2′-bis(diphenylphosphino)-1,1′-binaphthyl, or (S)-2,2′-bis(diphenylphosphino)-1,1′-binaphthyl.

2. The process according to claim 1, wherein the complex of Ru-BINAP is formed from a mixture of [Ru(COD)X₂]ₙ and BINAP before the complex is added to the reaction mixture, wherein COD is 1,5-cyclooctadiene, X is a halogen, and n is a natural number.

3. The process according to claim 1, wherein the complex of Ru-BINAP is generated in situ from a mixture of [Ru(COD)X₂]ₙ and BINAP in the reaction mixture, wherein COD is 1,5-cyclooctadiene, X is a halogen, and n is a natural number.

4. The process according to claim 1, wherein the reaction mixture further comprises a C₁-C₆ alcohol or a C₁-C₆ fluorinated alkyl alcohol.

5. The process according to claim 4, wherein the reaction mixture further comprises an acid.

6. The process according to claim 5, wherein the acid is selected from the group consisting of acetic acid, chloroacetic acid, propionic acid, and methanesulfonic acid.

7. The process according to claim 1, further comprising maintaining the reaction at a temperature of greater than 50° to about 140° C.

8. The process according to claim 1, wherein the ratio between the two enantiomers of the 3-amino-4-(2,4,5-trifluorophenyl)butanoic acid alkyl ester obtained is about 60% to about 100%, wherein the predominant enantiomer is the R enantiomer.

9. The process according to claim 1, wherein the ratio between the two enantiomers of the 3-amino-4-(2,4,5-trifluorophenyl)butanoic acid alkyl ester obtained is about 80% to about 100%, wherein the predominant enantiomer is the R enantiomer.

10. The process according to claim 1, wherein R′ is a hydrogen atom.

11. The process according to claim 10, further comprising converting 3-amino-4-(2,4,5-trifluorophenyl)butanoic acid alkyl ester to 3-amino-protected-4-(2,4,5-trifluorophenyl)butanoic acid alkyl ester.

12. The process according to claim 10, further comprising converting 3-amino-4-(2,4,5-trifluorophenyl)butanoic acid alkyl ester to Sitagliptin or salts thereof.

13. The process according to claim 1, wherein R′ is a C₁-C₄ alkoxycarbonyl, a C₁-C₄ haloalkoxycarbonyl, a C₆-C₂ benzyloxycarbonyl, or tert-butoxycarbonyl (BOC).

14. The process according to claim 1, wherein the chiral catalyst is a complex of Ru and a derivative of BINAP.

15. A process for preparing 3-amino-4-(2,4,5-trifluorophenyl)butanoic acid alkyl ester, comprises: preparing a mixture of a reducing reagent, 3-amino-4-(2,4,5-trifluorophenyl)but-2-enoic acid alkyl ester, and a chiral organic acid; and maintaining the mixture to obtain 3-amino-4-(2,4,5-trifluorophenyl)butanoic acid alkyl ester.

16. The process according to claim 15, wherein the reducing reagent is selected from a group consisting of sodium borohydride, sodium cyanoborohydride, lithium borohydride and lithium aluminum hydride.

17. The process according to claim 15, wherein the chiral organic acid is (R or S)-mandelic acid.

18. The process according to claim 15, wherein the mixture further comprises an ether.

19. The process according to claim 18, wherein the ether is a C₄ to C₈ alkyl ether or a C₄ to C₈ cyclic ether.

20. The process according to claim 15, wherein the ratio between the two enantiomers of the 3-amino-4-(2,4,5-trifluorophenyl)butanoic acid alkyl ester obtained is about 80% to about 100%, wherein the predominant enantiomer is the R enantiomer.

21. The process according to claim 15, further comprising converting 3-amino-4-(2,4,5-trifluorophenyl)butanoic acid alkyl ester to Sitagliptin or salts thereof.

22. A compound having the following formula: 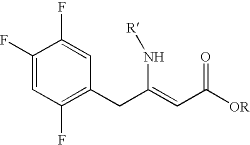
wherein R is a C₁-C₆ alkyl, or C₆-C₁₂ aryl, and R′ is a C₁-C₄ alkoxycarbonyl, a C₁-C₄ haloalkoxycarbonyl, a C₆-C₁₂ benzyloxycarbonyl, tert-butoxycarbonyl (BOC), trityl, F-MOC, or a carbamate having the formula of —CO₂R² (CBZ, R²═Bn), —SO₂R³, or —PO(R³)₂, wherein R³ is an alkyl, an aryl, or an alkylaryl.

23. The compound of claim 22, wherein the compound is isolated.

24. The compound of claim 22, wherein R′ is tert-butoxycarbonyl (BOC).

25. The process according to claim 1, further comprising converting the carbonyl group of 3-oxo-4-(2,4,5-trifluorophenyl)butanoic acid alkyl ester into a protected enamine functional group, producing the 3-amino-4-(2,4,5-trifluorophenyl)but-2-enoic acid alkyl ester.

26. The process according to claim 25, wherein R′ is tert-butoxycarbonyl (BOC), the process comprising: preparing a mixture of tert-butyl carbamate and 3-oxo-4-(2,4,5-trifluorophenyl)butanoic acid alkyl ester; and maintaining the mixture to obtain 3-tert-butoxycarbonylamino-4-(2,4,5-trifluorophenyl)but-2-enoic acid alkyl ester.

27. The process according to claim 26, wherein the mixture further comprises an organic acid that is selected from the group consisting of p-toluenesulfonic acid, methansulfonic acid, and trifluoroacetic acid.

28. The process according to claim 26, wherein the mixture further comprises an organic solvent that is a C₆-C₁₂ aromatic solvent or a halogenated C₁-C₆ alkane.

29. The process according to claim 28, wherein the organic solvent is selected from the group consisting of benzene, toluene, chlorobenzene, and methylene chloride.

30. The process according to claim 26, further comprising converting 3-tert-butoxycarbonylamino-4-(2,4,5-trifluorophenyl) but-2-enoic acid alkyl ester to Sitagliptin or salts thereof.

31. The process according to claim 26, further comprising: reacting 3-tert-butoxycarbonylamino-4-(2,4,5-trifluorophenyl)butanoic acid with 3-(trifluoromethyl)-5,6,7,8-tetrahydro[1,2,4]triazolo[4,3-a]pyrazine hydrochloride to obtain 4-(3-(trifluoromethyl)-5,6-dihydro-[1,2,4]triazolo[4,3-a]pyrazin-7(8H)-yl)-1-(2,4,5-trifluorophenyl)-4-oxobutan-2-yl-carbamate; and then removing the amino protected group in 4-(3-(trifluoromethyl)-5,6-dihydro-[1,2,4]triazolo[4,3-a]pyrazin-7(8H)-yl)-1-(2,4,5-trifluorophenyl)-4-oxobutan-2-yl-carbamate to obtain Sitagliptin.
